(19) United States
(12) Patent Application Publication
(10) Pub. No.: US 2022/0079977 A1
(43) Pub. Date: Mar. 17, 2022

(54) ELECTROLYZED WATER, METHOD FOR OBTAINING SAME AND USES OF SUCH A WATER FOR TREATING DISORDERS RELATING TO CELL SENESCENCE

(71) Applicant: WATERDIAM GROUP LLC, Miami, FL (US)

(72) Inventors: Anthony GINTER, Miami, FL (US); Laurent PUPUNAT, Delemont (FR)

(21) Appl. No.: 17/424,458

(22) PCT Filed: Jan. 17, 2020
(86) PCT No.: PCT/EP2020/051200

§ 371 Date: Jul. 20, 2021

(30) Foreign Application Priority Data
Jan. 20, 2019 (FR) 1900482

(51) Int. Cl. A61K 33/00; A61J 1/10; C12N 5/00; C02F 1/461

(52) U.S. Cl. CPC A61K 33/00; A61J 1/10; C12N 5/0018; C02F 2103/026; C02F 2001/46142; C02F 2001/46147; C02F 1/46109

(57) ABSTRACT

The present invention relates to a method for obtaining electrolysed water, the method comprising the following steps: —adding conductive c salt at a concentration of 0.5 to 2 g/L to untreated water —electrolysing the water obtained in i) using an electrolysis module comprising at least one boron-doped diamond electrode attached to a substrate of silicon in which the boron concentration is between 200 ppm (3×10¹⁹ B atoms/cm³) and 1500 ppm (2×1²⁰ B atoms/cm³), the duration of electrolysis being between 1 and 30 minutes. The invention also relates to a water obtained according to said method for treating diseases or disorders relating to cell senescence and to a composition containing such a water, a method for increasing the senolitic character of senescent cells and a device for treating skin conditions which comprises an infusion vessel or hag containing a water obtained according to the method of the invention.

The invention relates to the field of the treatment of the diseases or disorders relating to cell senescence, in particular the treatment of cancer or neurodegenerative diseases. The invention relates more particularly to a method for obtaining an electrolyzed water useful for the treatment of the diseases or the disorders relating to the cell senescence.

The invention also relates to the water obtained by this method, its use for the treatment of the diseases or the disorders relating to the cell senescence by direct application or in the form of compositions, and a suitable device for treating said conditions. The invention also relates to a method for selecting non-senescent cells useful in graft applications

As first described by Leonard Hayflick in 1961 when studying primary fibroblasts (WI-38) in culture, the fibroblasts proliferated for a limited number of population doublings before remaining irreversibly stuck in a growth arrest plateau. It is this phenomenon that he will call the cell senescence. This plateau, called the Hayflick limit or replicative senescence plateau, is observed for most normal cells (e.g. fibroblasts, endothelial cells, etc.).

The cell senescence is generally understood as a definitive cell cycle arrest resulting from a variety of causes. The arrest mechanism of the cell cycle induced by the cell senescence is complex and involves interactions between the shortening of the telomeres, the inflammation and the cellular stress.

By targeting the cell senescence, because of its impact on diseases such as cancer or age-related neurodegenerative diseases, researchers believe that they have a promising avenue for combating these diseases, whereas the conventional therapeutic arsenal, which is not without side effects, remains almost ineffective if we consider, for example, the Alzheimer's disease.

Reference is made to US2017198253, which relates to a method for selectively killing senescent cells and for treating diseases and disorders associated with the senescence by administering a senolitic agent. The diseases and the disorders associated with the senescence that can be treated by the methods using the senolitic agents described herein comprise the cardiovascular diseases and disorders associated with or caused by an arteriosclerosis, such as the atherosclerosis; the idiopathic pulmonary fibrosis; the chronic obstructive pulmonary disease; the osteoarthritis; the senescence-associated ophthalmic diseases and disorders; and the senescence-associated dermatologic diseases and disorders. The senolitic agents are comprised among the MDM2 inhibitors such as the cis-imidazoline derivatives or inhibitors of Bcl-2, Bcl-xL, or p-Akt proteins.

Reference is also made to WO2018215795 which relates to an agent intended to be used for selectively killing one or more senescent cells, said agent being chosen from the following: a cardiac glycoside or aglycone, an inhibitor of the focal adhesion kinase (FAK), an inhibitor of the HMG-CoA reductase, JF D00244, cyclosporine, tyrphostin AG879, cantharidin, diphenyleiodonium chloride, rottlerin, 2,3-dimethoxy-1,4-naphthoquinone, LY-367, 265, rotenone, idarubicin, dequalintum chloride, vincristine, nitazoxanide, nitrofurazone, temsirolimus, eltrombopag, adapalene, azacyclonol, enoxacin and raltegravir, and pharmaceutically suitable salts thereof. Another aspect relates to compounds intended to be used in the treatment or the prevention of a disease or a disorder associated with the senescence, and the related methods.

Also referenced is the application US2018256568 which relates to a method for treating a disease or a disorder in a target tissue in a subject, wherein the symptoms of the disease or the disorder are caused at least partly by senescent cells located in or around the target tissue, wherein the target tissue is free of cancer, and wherein the senescent cells are defined as cells expressing the p16 protein.

The method comprises administering into or around the target tissue of a series of treatments with a pharmaceutical composition that comprises a senolitic agent that comes into contact with the senescent cells located in or around the target tissue and selectively removes this senescent cells without affecting adjacent non-senescent cells, if possible. The application describes a series of chemical molecules allowing to treat the senescent cells.

Although effective, these solutions have a major disadvantage in that these chemicals are not without toxicity or side effects. Of course, they allow to affect the senescent cells, but we cannot exclude that they also affect the healthy cells or tissues. A lack of selectivity relating to the use of chemicals that may have multiple protein targets is not without risk.

In the case of the grafts, whether of skin or liver for example, the removal of the senescent cells would allow, during the transplantation or the graft, to avoid the patient having, in addition to healthy grafted or transplanted cells, senescent cells which, in the long term, could induce diseases due to their presence. This is also a major challenge. Again, a selection of the right cells or of a healthy tissue free of senescent cells through the use of chemicals for the graft or the transplantation of a tissue is not without problems of side effects or toxicity on the healthy cells or tissue.

Thus, there is a real need for alternative methods or compositions allowing to selectively remove or effectively treat the senescent cells and the diseases associated with the presence of this cells while reducing the side effects of this treatment methods as well as selectively affecting the senescent cells with little or no effect on the healthy cells of a tissue or an organ.

One of the aims of the invention is therefore to remedy the aforementioned disadvantages and to meet the aforementioned needs by providing an electrolyzed water for the treatment of the disorders relating to the cell senescence.

More particularly, one of the objects of the invention is the envisaged use of the electrolyzed water according to the invention in the treatment of diseases relating to cell senescence, such as in particular cancers or neurodegenerative diseases.

The electrolyzed water according to the invention is obtained by the implementation of a three-step method which starts with using of a running or spring water optionally added with a sulfate salt, carbonates or any other compatible salt known to the person skilled in the art at the concentration 0.5 to 2 g/L followed by electrolyzing said water by an electrolysis module comprising at least one boron-doped diamond electrode attached to a substrate of silicon in which the boron concentration is between 200 (3×10¹⁹ B atoms/cm³) and 2000 ppm (3.52×10²⁰ B atoms/cm³), in particular between 200 ppm (3×1019 B atoms/cm3) and 1500 ppm (2×1020 B atoms/cm3), said module subjecting the water to a quantity of current during the electrolysis process of less than 500 mAh/L of water, more preferably comprised between 40 and 250 mAh/L of water, even more preferably between 50 and 200 mAh/L, the duration of the electrolysis being between 1 minute and 60 minutes.

The electrolysis may be performed at one time, continuously, or cyclically with a number of cycles being comprised between 2 and 12 cycles over a 24 hour period, with two consecutive cycles being at least 30 minutes apart.

The invention also relates to an electrolyzed water obtained by the method described above.

The invention also relates to a method for increasing the senolitic character of senescent biological cells present in a cell medium, characterized in that it consists in introducing into the cell medium an electrolyzed water obtained according to the method previously described. This method consists particularly in increasing at least the beta-galactosidase (SA-β-Gal) activity associated with the senescence by bringing into contact said cells with the electrolyzed water thus obtained. The inventors have indeed discovered that the electrolyzed water obtained according to the invention had the singular property of significantly increasing this beta-galactosidase (SA-β-Gal) activity when senescent cells such as those present in particular in fibroblasts were brought into contact with said water.

Advantageously according to this method, the percentage of water contained in the cell medium is strictly greater than 0, and preferably greater than 5%, and even more preferably greater than 25%.

Advantageously, according to the method for increasing the senolitic character of the invention, the senescent cells are maintained in the cell medium in contact with water for a period of more than 1 min.

Such an increase in the senolitic character of senescent cells has the major advantage of allowing an induced selection of healthy cells with respect to the senescent cells by accelerating the natural process of senescence in a cell medium or biological tissue, and thus the accelerated disappearance of said cells in favor of healthy cells. This opens up exceptional possibilities for using the water of the invention for prophylactic purposes, as an aid to therapeutic treatment, or even as a direct therapeutic treatment.

The invention also relates to a composition comprising an electrolyzed water obtained according to the method described above for use as a medicament in the treatment of disorders relating to the senescence.

Preferably, the disorders targeted by the inventive use comprise the diseases related to the senescence and in particular certain cancers or the neurodegenerative diseases.

The composition comprising electrolyzed water according to the invention consists of 95 to 100% of electrolyzed water and 0 to 5% of an excipient and/or an emulsifier.

The composition according to the invention may be in the form of a cream, a gel, a wound dressing or even a patch.

Furthermore, when the composition according to the invention can be composed exclusively of electrolyzed water, the latter can be added to a device containing it, such as an infusion reservoir or bag containing the pure electrolyzed water or in the form of a liquid composition.

The electrolyzed water is obtained by means of an electrolysis module allowing the implementation of the method described above, said module being able to be present in a fixed or mobile manner in a device such as a reservoir, said reservoir being able to be used to fill infusion bags on a production line for example.

It may also be present in a conventional water distribution circuit, preferably at the outlet of a distribution system such as a tap, if one considers preparing a drink from electrolyzed water, which may then be consumed for the purpose of treating the disorders relating to the cell senescence by oral route.

The invention also relates to a method of selecting non-senescent cells in a cell extract or a biological tissue comprising a plurality of cells by bringing them into contact with an electrolyzed water obtained by the implementation of the method according to the invention for a period of 1 to 30 minutes.

Thus, obtaining a cell extract or a healthy tissue free, in whole or in large part, of senescent cells, obtained by the implementation of the selection method described above, may be of interest in the treatment of the grafts, in particular of skin or liver.

The appended drawings illustrate the invention:

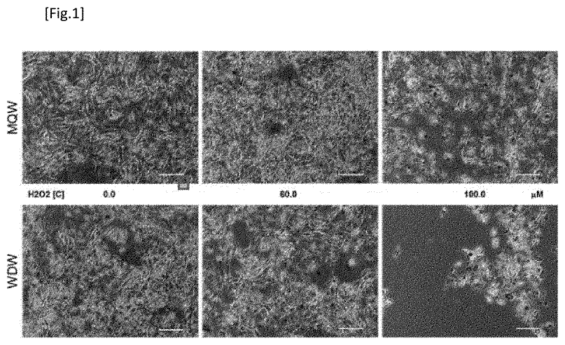

FIG. 1 shows the effectiveness of the electrolyzed water according to the invention named WDW on tumor cell lines of the chondrosarcoma type in comparison with an ultra-pure non-electrolyzed water MQW.

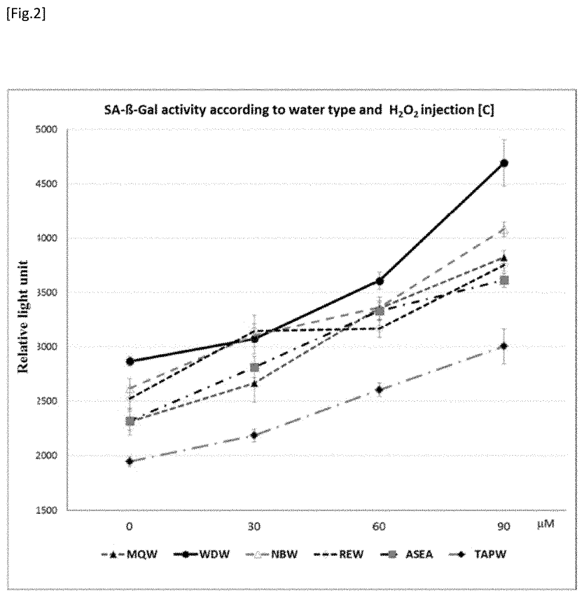

FIG. 2 shows the superiority of the electrolyzed water obtained according to the method of the invention, on the modification of the SA-β-Gal activity whose increase is witness to the presence of senescent cells.

The present invention will be described in more detail with the aid of one or more examples which in no way limit the invention.

In biology, the senescence is a complex process that leads to a slow degradation of the functions of the cell at the origin of the aging of the organisms. This is a complex biological mechanism, involving the degradation of many genes, especially in the senescent cells. It is usually the telomeric part of the chromosomes that is affected.

A telomere is a highly repetitive region of DNA present at the end of a chromosome. Each time a rod chromosome of a eukaryote is replicated, during the mitosis also called cell division, the enzymatic complex of the DNA polymerase is unable to copy the last nucleotides. The telomeres shorten with age, presumably as a result of inflammation and/or repeated stresses that the organisms undergo. This results in the appearance of diseases such as age-related cancer or neurodegenerative diseases in an organism with a large number of senescent cells.

One of the main characteristics of the senescent cells is that they cease to divide in contrast to the normal cells of the organism, but the fact that these cells no longer divide also means that the mode of operation of these cells differs from healthy or normal cells.

They also actively modify their environment by secreting cytokines, growth factors as well as proteases of the extracellular matrix.

This phenomenon plays an important role in the inflammatory response, in the remodeling of the formation of the extracellular matrix and in the maintenance of the senescent state which will end in the course of time by a tissue degeneration.

In some cases, the loss of proliferation competent cells may be responsible for a pathology, as suspected in the glaucoma, the cataracts, the pancreatic diabetes or the osteoarthritis.

In other cases, the inflammation caused by the SASP (Senescence-Associated Secretory Phenotype) may play a causal role in the appearance of diseases; the senescent cells with a high inflammatory potential will gradually enter in the tissue environment where they will in a way “contaminate” healthy cells in the vicinity, which in turn will become senescent, as it is suspected in the case of diseases such as the atherosclerosis, the cardiovascular disease as well as the age-related cancers.

Similarly, in the case of grafting or transplantation of tissue such as the skin or the liver, or even of an organ, the presence of senescent cells with significant inflammatory power may limit the effectiveness of the graft on a subject who needs a graft with the “healthiest” tissue possible (free of as many senescent cells as possible).

The present invention concerning a particular electrolyzed water, obtained by a method involving particular boron-doped diamond electrodes attached to silicon, allows to solve, at least partially, the problems or disadvantages previously mentioned.

The electrolysis module allowing to implement the method of preparing electrolyzed water according to the invention comprises at least one, preferably at least two boron-doped diamond electrodes which are attached to a substrate of silicon.

The active or contact surface of each electrode is advantageously comprised between 10 and 100 cm2, preferably 60 and 80, more preferably about 70 cm2.

The boron doping of the diamond electrode also has an impact on the properties of the water obtained; the boron concentration is between 200 ppm (3×10¹⁹ B atoms/cm³) and 1500 ppm (2×10²⁰ B atoms/cm³).

This boron concentration as well as the nature of the electrodes based on silicon diamond gives it properties allowing it to operate on a potential between −1V and −2V on the cathodic polarization and on +2V and +4V on the anodic polarization compared to a hydrogen reference electrode.

The electrolysis module is connected to a power supply module and is open to a flow of water which will pass through it. In order to operate properly and not to compromise the correct working conditions, the electrodes are supplied with direct current by the power supply module which is connected to the electrolysis module supplying a direct current to the electrode or the electrodes.

Generally the supply current is set between 1.5 A and 7 A. If polarity reversal is required, this operation can be done automatically by the power supply module.

The water may come from different sources but must pass through the electrolysis module as the module cannot operate in the absence of water. The electrolysis module can be permanently traversed by water depending on the device in which it is located. An internal measuring system comprising a hydraulic flow sensor is present, interacting with the power module and that allow the electrolysis module to be put on standby, switched off or switched on in the absence or presence of water, and thus to activate the electrolysis.

The water electrolysis module according to the present invention can advantageously be operated in automatic mode or can be activated or deactivated on demand, manually or by means of a remote control system.

A further advantage of the present invention is that the water electrolysis module does not necessarily need to be permanently activated, but can usefully be activated cyclically, i.e., at convenient intervals, preferably but not necessarily at regular intervals. It has been found that the electrolysis of water at regular intervals allows the water to remain therapeutically active for a long time.

In particular, electrolysis cycles can be performed at a rate of 2 to 12 cycles over a period of 24 hours, with two consecutive cycles at least 30 minutes apart, thus allowing to obtain a water with a therapeutic potential.

The method for preparing electrolyzed water according to the invention using the module described above comprises the following steps:

a. providing a running or spring water with the optional addition of a conductivity salt at a concentration of 0.5 to 2 g/L

b. performing an electrolysis of the water thus provided by a device comprising at least one boron-doped diamond electrode attached to a substrate of silicon in which the concentration of boron is between 200 ppm (3×10¹⁹ B atoms/cm³) and 1500 ppm (2×10²⁰ B atoms/cm3).

Advantageously, the amount of current delivered during the electrolysis process is between 15 and 500 mAh/L of water, more preferably 40 to 250 mAh/L of water, still more preferably 50 to 200 mAh/L; the electrolysis time being comprised between 1 and 60 minutes.

The method of the invention thus allows to obtain a water suitable for use in the treatment of the diseases or the disorders relating to cell senescence.

As mentioned above, the treatment of the diseases or the disorders relating to the cell senescence can, without being limiting, comprise the treatment of the age-related diseases such as the neurodegenerative diseases, the skin conditions, but also the glaucoma, the cataracts, the pancreatic diabetes, the osteoarthritis, the atherosclerosis, the cardiovascular diseases or the age-related cancers. The water obtained according to the method of the invention can also be useful in the case of grafting or transplantation of tissue such as the liver or an organ or the skin graft.

The resulting water may also be used in different forms or compositions or within different devices.

The devices used may be selected from an infusion reservoir or bag comprising the electrolyzed water such as described above. The electrolysis module used will then be integrated into an existing water circuit in order to prepare the electrolyzed water; it is also possible to imagine the electrolysis module connected to or immersed in a reservoir.

Any water, whether natural, spring water or municipal water, may be used in the method for obtaining electrolyzed water according to the invention.

The advantage and the flexibility of use of the electrolysis module according to the invention allows to obtain, from an abundant source such as water, a less expensive treatment because it does not require conventional chemicals in the treatment of the diseases or disorders relating to the senescence, while avoiding the side effects of said chemicals.

The electrolyzed water obtained by the method according to the invention can also be present in more conventional compositions which are found on sale in the pharmacies or the parapharmacies or even the supermarkets, said composition comprising 95 to 100% of electrolyzed water and 0 to 5% of an excipient and/or emulsifier.

Said composition is for use as a medicament in the treatment of diseases or disorders relating to the cell senescence, most particularly the age-related cancers or the neurodegenerative diseases.

It may be in the form of a cream, a gel, a wound dressing, a patch, when it is a question, for example, of removing the senescent cells from the skin and maintaining the healthiest possible skin comprising a maximum of healthy or non-senescent cells.

Another case that may be considered more particularly is that of the grafts of skin cell, tissue or even organ such as the liver, for example, is the use of the electrolyzed water according to the invention in a cell, tissue or organ culture medium in vitro.

This culture medium usually comprises, in addition to the electrolyzed water according to the invention, all the nutrients essential for maintaining its activity, such as growth factors, nutrients such as the amino acids, salts, additives possibly animal serum, and other compounds such as sugars or antibiotics in order to avoid the contamination of the culture medium, as well as others well known to the person skilled in the art in the field of the culture media.

Another potential dosage form for the composition according to the invention may be an aqueous gel also called hydrogel. A hydrogel is a gel in which the swelling agent is the water electrolyzed according to the method of the invention. The matrix of a hydrogel is typically a network of polymers that are insoluble in water, but are capable of substantial swelling in the presence of a large amounts of water or aqueous solutions.

Two examples of biological tests validating the potential of the electrolyzed water on the senescent cells in the presence of healthy cells have been performed.

As shown in FIGS. 1 and 2, trials were carried out in order to validate the potential of the electrolyzed water according to the invention.

FIG. 1 represents the results obtained during comparative trials between a water according to the invention WDW, obtained according to the implementation of the method according to the invention under a current of 62.5 mAh/L, and an ultrapure water MQW which is obtained with filters from the company Millipore, in the presence of variable concentrations of H2O2, the two waters being applied to cultures of chondrosarcoma cells of cell line JJ012. The cell culture medium is of the EMEM type obtained by mixing powders (Thermo Fisher, GIBCO #41500-034, Penicylin Steptomycin (PenStrep) #15140122, and 10% Fetal Bovine Serum (FBS) #26140079) and sterilized and filtered (0.2 μm filter) and containing said cells.

It is apparent that the disappearance of the senescent cells is greater in the presence of WDW water, showing that it is an effective means of selecting non-senescent cells with respect to the presence of senescent cells in a cell culture medium comprising both non-senescent and senescent cells.

In FIG. 2, it is assumed that normal cells can permanently lose their ability to proliferate when subjected to stress, a process called cell senescence. The senescence-associated beta-galactosidase (SA-β-Gal) activity, detectable at pH 6.0, allows the identification of senescent cells in cultured and mammalian tissues. The activity was measured with the BioVision kit (catalogue no K821-100). The beta-galactosidase (β-Gal, EC: 3.2.1.23) is an enzyme that hydrolyses the β-galactosides to monosaccharides. The senescence-associated β-Gal (SA-β-Gal) is an isoform of the β-Gal that exhibits the optimal activity at pH 6.0 and is primarily used as a biomarker for the senescent cells (K802). The detection is based on the hydrolysis by the beta-galactosidase of a non-fluorescent substrate to generate a strongly fluorescent product. The fluorescein produced (3′, 6′-dihydroxyspiro [isobenzofuran-1 (3H), 9′-[9H] xanthein]-3-one) can then be measured (substrate-fluorescence→Galactose+fluorescein (Ex/Em=480/530 nm). The test was performed 36 hours after the induction of H₂O₂ as measurements at 530 nm.

It is deduced that in the presence of different waters with increasing levels of H₂O₂:
electrolyzed water obtained according to the implementation of the method according to the invention (WDW),
ultrapure water obtained with filters from the company Millipore (MQW),
electrolyzed water obtained with a Niobium electrode (NBW),
electrolyzed water with a single electrode (REW),
running water (TAPW) and
commercial water of the company ASEA called “redox signaling water” or ASEA@ REDOX.

Using the SA-β-Gal activity as an indication/biomarker of the cell senescence, the experiment described in FIG. 2 confirms that:

1) The electrolyzed water obtained by the implementation of the method according to the invention (WDW) is the most effective in terms of elevation of the cell senescence than other electrolyzed or commercial waters and this when the concentration of H2O2 is 0 μM corresponding to the control.

2) The electrolyzed water WDW is more efficient than the other waters used for all concentrations of H2O2 used.

3) This also proves that the waters used, although mainly composed of water molecules H2O, do not have the same properties because for the same type of cells grown under the same conditions, their impact on the SA-β-Gal activity is not the same. Thus, for WDW, it is indeed a specific water that can be considered as a product obtained by the implementation of its production method.

The examples of images and devices presented according to the invention, as well as the various embodiments mentioned, in no way limit the scope of the invention as claimed; they are given by way of illumination in order to better understand the invention.

1. A method for obtaining an electrolyzed water comprising:
electrolyzing a running or spring water, optionally comprising a conductivity salt, e.g. NaCl at a concentration of 0.5 to 2 g/L with an electrolysis module comprising at least one boron-doped diamond electrode attached to a substrate of silicon wherein a boron concentration of the electrode is between 200 (3×10¹⁹ B atoms/cm³) and 2000 ppm (3.52×10²⁰ B atoms/cm³), in particular between 200 ppm (3×10¹⁹ B atoms/cm³) and 1500 ppm (2×10²⁰ B atoms/cm³),
subjecting the water by said module to a quantity of current during the electrolysis comprising between 15 and 500 mAh/L of water, more preferably 40 to 250 mAh/L of water, still more preferably 50 to 200 mAh/L, a duration of the electrolysis being between 1 minute and 60 minutes.

2. The method according to claim 1, wherein the electrolysis is performed either continuously or cyclically, a number of cycles being comprised between 2 and 12 cycles per 24 hour period, each electrolysis cycle being spaced from a preceding or following cycle by at least 30 minutes.

3. Water obtained by:
electrolyzing a running or spring water, optionally comprising a conductivity salt, e.g. NaCl at a concentration of 0.5 to 2 g/L with an electrolysis module comprising at least one boron-doped diamond electrode attached to a substrate of silicon wherein a boron concentration of the electrode is between 200 (3×10¹⁹ B atoms/cm³) and 2000 ppm (3.52×10²⁰ B atoms/cm³), in particular between 200 ppm (3×10¹⁹ B atoms/cm³) and 1500 ppm (2×10²⁰ B atoms/cm³),
subjecting the water by said module to a quantity of current during the electrolysis comprising between 15 and 500 mAh/L of water, more preferably 40 to 250 mAh/L of water, still more preferably 50 to 200 mAh/L, a duration of the electrolysis being between 1 minute and 60 minutes.

4. A method for increasing the senolitic character of senescent biological cells present in a cell medium, consisting of characterized in that it consists in introducing into the cell medium a water obtained by:
electrolyzing a running or spring water, optionally comprising a conductivity salt, e.g. NaCl at a concentration of 0.5 to 2 g/L with an electrolysis module comprising at least one boron-doped diamond electrode attached to a substrate of silicon wherein a boron concentration of the electrode is between 200 (3×10¹⁹ B atoms/cm³) and 2000 ppm (3.52×10²⁰ B atoms/cm³), in particular between 200 ppm (3×10¹⁹ B atoms/cm³) and 1500 ppm (2×10²⁰ B atoms/cm³),
subjecting the water by said module to a quantity of current during the electrolysis comprising between 15 and 500 mAh/L of water, more preferably 40 to 250 mAh/L of water, still more preferably 50 to 200 mAh/L, a duration of the electrolysis being between 1 minute and 60 minutes.

5. The method according to claim 4, wherein the percentage of water contained in the cell medium is strictly greater than 0, and preferably greater than 5%, more preferably greater than 25%.

6. The method according to claim 4, wherein the senescent cells are maintained in the cell medium in contact with the water for a period of more than 1 min.

7.-8. (canceled)

8. A composition comprising an electrolyzed water for use as a medicament in the treatment of the disorders relating to the cell senescence, in particular the treatment of the cancer or the neurodegenerative diseases, the composition comprising:
(A) 95 to 100% of electrolyzed water obtained by:
electrolyzing a running or spring water, optionally comprising a conductivity salt, e.g. NaCl at a concentration of 0.5 to 2 g/L with an electrolysis module comprising at least one boron-doped diamond electrode attached to a substrate of silicon wherein a boron concentration of the electrode is between 200 (3×10¹⁹ B atoms/cm³) and 2000 ppm (3.52×10²⁰ B atoms/cm³), in particular between 200 ppm (3×10¹⁹ B atoms/cm³) and 1500 ppm (2×10²⁰ B atoms/cm³),
subjecting the water by said module to a quantity of current during the electrolysis comprising between 15 and 500 mAh/L of water, more preferably 40 to 250 mAh/L of water, still more preferably 50 to 200 mAh/L, a duration of the electrolysis being between 1 minute and 60 minutes and
(B) 0 to 5% of an excipient and/or emulsifier.

9. (canceled)

10. A device for treating the diseases or disorders relating to the cell senescence comprising:
an infusion bag containing a composition comprising:
(A) 95 to 100% of electrolyzed water obtained by:
electrolyzing a running or spring water, optionally comprising a conductivity salt, e.g. NaCl at a concentration of 0.5 to 2 g/L with an electrolysis module comprising at least one boron-doped diamond electrode attached to a substrate of silicon wherein a boron concentration of the electrode is between 200 (3×10¹⁹ B atoms/cm³) and 2000 ppm (3.52×10²⁰ B atoms/cm³), in particular between 200 ppm (3×10¹⁹ B atoms/cm³) and 1500 ppm (2×10²⁰ B atoms/cm³),
subjecting the water by said module to a quantity of current during the electrolysis comprising between 15 and 500 mAh/L of water, more preferably 40 to 250 mAh/L of water, still more preferably 50 to 200 mAh/L, a duration of the electrolysis being between 1 minute and 60 minutes and
(B) 0 to 5% of an excipient and/or emulsifier.
